(19) United States
(12) Patent Application Publication
(10) Pub. No.: US 2012/0122919 A1
(43) Pub. Date: May 17, 2012

(54) PHARMACEUTICAL COMPOSITION COMBINING TENATOPRAZOLE AND A HISTAMINE H2-RECEPTOR ANTAGONIST

(75) Inventors: François Schutze, Saint-Nom-La-Breteche (FR); Suzy Charbit, Creteil (FR); Hervé Ficheux, Nogent-sur-Marne (FR); Michel Homerin, Courcouronnes (FR); Alain Taccoen, Le Chesnay (FR); Nathalie Taccoen, La Chesnay (FR); Yoshio Inaba, Tokyo (JP)

(73) Assignees: MITSUBISHI PHARMA CORPORATION, Tokyo (JP); SIDEM PHARMA, Luxembourg (LU)

(21) Appl. No.: 13/297,122

(22) Filed: Nov. 15, 2011

Related U.S. Application Data
(63) Continuation of application No. 10/532,114, filed on Jun. 23, 2006, now abandoned.

(30) Foreign Application Priority Data
Oct. 21, 2002 (FR) 02/13114

(51) Int. Cl. A61K 31/437; A61P 1/04

(52) U.S. Cl. 514/303

(57) ABSTRACT

The invention relates to a novel pharmaceutical combination. The inventive pharmaceutical composition, which is intended for the treatment of pathologies linked to gastric hyperacidity, comprises a combination of tenatoprazole and one or more histamine H2-receptor antagonists preferably selected from cimetidine, ranitidine, famotidine and nizatidine. The invention is particularly suitable for the treatment of duodenal and gastric ulcers and the symptoms of, and lesions caused by, gastroesophageal reflux.

### RELATED APPLICATIONS

The present application is a continuation of U.S. patent application Ser. No. 10/532,114 filed Jun. 23, 2006, the contents of which are hereby incorporated by reference in their entirety.

### BACKGROUND OF THE INVENTION

1. Field of the invention

The present invention concerns a new drug combination, and more particularly a new pharmaceutical composition combining an histamine H2-receptor antagonist and tenatoprazole, for the treatment of diseases related to gastric hyperacidity, particularly gastric and duodenal ulcers, and symptoms and lesions related to gastroesophageal reflux.

2. Description of the Related Art

When treating digestive disorders such as dyspepsia, gastric hyperacidicity, gastritis, etc., the aim is usually to eliminate the gastric acid which is responsible for damaging the gastric mucosa. Various medicinal products, such as antacids, histamine H2-receptor antagonists and proton pump inhibitors have been used for such treatments.

Thus histamine H2-receptor antagonists are frequently employed to treat disorders linked to the hypersecretion of gastric acid, for example the treatment of gastric ulcers, as they inhibit the secretion of gastric acid. Histamine H2-receptor antagonists may be chosen from a series of well-known products such as cimetidine, ranitidine, famotidine, etc.

Proton pump inhibitors have also proved their usefulness in the treatment of gastric and duodenal ulcers. The first known derivative of this series was omeprazole, described in Patent No. EP 005.129, and endowed with properties which inhibit the secretion of gastric acid and is widely employed as an anti-ulcerative in human therapeutics. Other proton pump inhibitors include rabeprazole, pantoprazole and lansoprazole, which all exhibit structural analogy and belong to the group of pyridinyl-methyl-sulfinyl-benzimidazoles. Tenatoprazole has a similar structure, but of the imidazopyridine type. These compounds are sulfoxides presenting with asymmetry at the level of the sulphur atom, and therefore generally take the form of a racemic mixture of two enantiomers.

Omeprazole has also been envisaged for the treatment of gastroesophageal reflux disorders, but its action in this indication is not entirely satisfactory. Thus studies have shown that its duration of action, like that of other proton pump inhibitors, is insufficient to ensure the efficient treatment of nocturnal reflux.

Tenatoprazole, or 5-methoxy-2-[[(4-methoxy-3,5-dimethyl-2-pyridyl)methyl]sulfinyl]imidazo[4,5-b]pyridine, is described in Patent No. EP 254.588, together with its properties which inhibit ATPase (H⁺+K⁺) and gastric acid secretion.

Various combinations of active substances belonging to these categories have also been envisaged with the aim of improving their pharmacological effects or attenuating their known adverse effects. For example, U.S. Pat. No. U.S. 6,090,412 describes a pharmaceutical formulation for oral administration combining a histamine H2-receptor antagonist such as famotidine, with at least two standard antacids such as sodium bicarbonate and magnesium hydroxide, which display a strong neutralisation potential, and an aluminium hydroxide gel which exhibits weak neutralisation potential. Patent No. FR 2,656,528 describes the combination of cimetidine and an antimuscarinic agent, pirenzepine, which is presented as diminishing the adverse effects of cimetidine.

A study has shown that administering omeprazole twice a day and ranitidine in the evening to patients suffering from gastoesophageal reflux might be useful (Peghini P L, Katz P O, Castell D O, “Ranitidine controls nocturnal gastric acid breakthrough on omeprazole : a control study in normal subjects” Gastroenterology (1998) 115(6):1335-9) but other studies mention that a treatment comprising administering omeprazole in the morning and in the evening is more efficient than a treatment combining the administration of omeprazole with that of ranitidine (Cross L B, Justice L N, “Combination drug therapy for gastroesophageal reflux disease”, Ann. Pharmacother. (May 2002) 36(5):912-6). In view of such results, it can be considered that the association of a histamine H2-receptor antagonist and a proton pump inhibitor has no particular advantage, probably partly because of the low elimination half-life of the latter.

On the contrary, the studies performed by the applicant have shown that the combination of a specific proton pump inhibitor, i.e. tenatoprazole, and a histamine H2-receptor antagonist procures unexpected effects which compared with other proton pump inhibitors and other histamine H2-receptor antagonists, used alone or in combination. More particularly, it has been shown that the combination of tenatoprazole and one or more histamine H2-receptor antagonists enables control of gastric acidity which is markedly superior to that achieved with each of the components used alone, and particularly allows the effective treatment of patients suffering from symptoms and lesions related to gastroesophageal reflux and refractory to standard therapy with a proton pump inhibitor.

### SUMMARY OF THE INVENTION

The object of the present invention is therefore a pharmaceutical composition combining a specific proton pump inhibitor, tenatoprazole, with one or more histamine H2-receptor antagonists.

A further object of the present invention is a pharmaceutical composition for administration by mouth, comprising tenatoprazole and one or more histamine H2-receptor antagonists, in a form adapted to the treatment of diseases related to gastric hyperacidity, particularly gastric and duodenal ulcers, and the symptoms and lesions of gastroesophageal reflux.

Another object of the invention is the combined use of tenatoprazole and at least one histamine H2-receptor antagonist for the treatment of diseases related to gastric hyperacidity, particularly gastric and duodenal ulcers, and the symptoms and lesions of gastroesophageal reflux, as well as the combined use of tenatoprazole and at least one histamine H2-receptor antagonist for the manufacture of a medicinal product intended for the treatment of diseases related to gastric hyperacidity, particularly gastric and duodenal ulcers, and the symptoms and lesions of gastroesophageal reflux.

### DETAILED DESCRIPTION OF THE PREFERRED EMBODIMENTS

According to the invention, tenatoprazole can be used in a free or salt form, such as, for example, a potassium, magnesium, sodium or calcium salt.

The histamine H2-receptor antagonist employed in the composition of the invention may be selected from cimetidine, ranitidine, famotidine or nizatidine.

The ratio between the content in tenatoprazole and that of the histamine H2-receptor antagonist may be between 1:30 and 1:2, and preferably between 1:20 and 1:5; this ratio may vary as a function of the histamine H2-receptor antagonist chosen.

Previous studies have shown that amongst both patients suffering from symptoms and lesions of gastroesophageal reflux and healthy volunteer subjects, approximately 70% of them experienced a nocturnal peak of acidity, i.e. a pH below 4 for a period of at least one hour during the nocturnal period between 22 h00 and 06 h00. It is also known that the severity of oesophageal mucosal lesions is linked to the duration of exposure to a gastric pH lower than 4.

The new studies performed have shown that these symptoms and lesions can be treated effectively with a composition which complies with the present invention, combining tenatoprazole and a histamine H2-receptor antagonist, and that this advantage results from a type of specific activity of tenatoprazole which complements that of the histamine H2-receptor antagonist.

Indeed, tenatoprazole can be distinguished from other proton pump inhibitors by its astonishingly longer elimination half-life, and also its considerable degree of tissue exposure, as has been demonstrated during experiments conducted by the applicant.

Thus, the phase I study in Caucasian individuals (n=8 per group) made it possible to demonstrate the influence of different doses of tenatoprazole on pharmacokinetic parameters, in the case of the oral administration of a single dose and a daily dose for a period of 7 days.

The doses tested were 10, 20, 40 and 80 mg of tenatoprazole.

The results obtained are grouped in Table 1 below.

TABLE 1

| | Single dose | | | | Repeated dose (7 days) | | | |
|---|---|---|---|---|---|---|---|---|
| | 10 mg | 20 mg | 40 mg | 80 mg | 10 mg | 20 mg | 40 mg | 80 mg |
| Cmax (μg/ml) | 0.9 | 2.4 | 5.3 | 8.3 | 1.6 | 3 | 5.5 | 11.8 |
| Tmax (h) | 4 | 4 | 3 | 3 | 3 | 2 | 3 | 2 |
| T½ (h) | 5 | 6 | 6 | 7 | 5 | 8 | 9 | 9.2 |
| AUC 0-t | 8 | 24 | 43 | 97 | 13 | 36 | 75 | 218 |

In this table, the abbreviations employed have the following meaning:
Cmax maximum concentration
Tmax time required to attain maximum concentration
T½ elimination half-life
AUC₀₋ₜ area under the curve, between time 0 and the last measurable concentration.

The results shown in Table 1 above demonstrate that the mean elimination half-lives were between 5 and 6 hours after the administration of a single dose, and between 5 and 9.2 hours after administration for seven days, depending on the dose. Tenatoprazole also exhibited high AUC values (area under the curve), providing evidence of a low rate of metabolism and/or high bioavailability via the oral route. Furthermore, whatever the conditions of administration, single or repeated, the Cmax, AUC₀₋ₜ and AUC_{0-inf} values increased in a linear fashion. The AUC_{0-inf} value was calculated by extrapolation.

A comparison of AUC values between two proton pump inhibitors, lansoprazole and omeprazole, had already been made by Tolman et al. (J. Clin. Gastroenterol., 24(2), 65-70, 1997), but this did not enable a judgement as to the superiority of one product over the other. Indeed, different criteria must be taken into account, i.e. the time required for pump regeneration, the period above the minimum concentration necessary to inhibit proton pumps. With respect to the pump regeneration time, it is observed that pumps usually have a half-life of about 30 to 48 hours, and are therefore totally renewed every 72 to 96 hours.

The pharmacokinetic study performed by the claimant showed that, thanks to the unexpected pharmacokinetic properties described above, tenatoprazole could counteract the proton pump regeneration phenomenon by maintaining an inhibitory concentration for a sufficiently long period of time to meet the two criteria specified previously.

Thus, the prolonged exposure related to the long half-life of tenatoprazole, demonstrated by the AUC values obtained, endow it with a longer presence at the sites of activity and thus procure a pharmacodynamic effect which is prolonged over time. Experiments have thus shown that tenatoprazole is endowed with a plasma half-life/pump regeneration time ratio which is notably higher than that seen with other proton pump inhibitors, thus permitting its use in pathologies where currently available medicinal products have little effect, and particularly in the treatment of the nocturnal symptoms of gastroesophageal reflux and gastric and duodenal ulcers.

Therefore, when it is combined with a histamine H2-receptor antagonist, such as cimetidine or ranitidine, and preferably administered in the evening before going to bed, tenatoprazole, when compared with other proton pump inhibitors, procures a significant advantage with respect to suppressing gastric acidity, and consequently enables effective action on the nocturnal gastric acid peak and on the nocturnal symptoms of patients suffering from gastroesophageal reflux, in whom it enables marked relief, even in those refractory to standard therapies with commonly employed proton pump inhibitors such as omeprazole.

The composition of the invention also enables a marked advantage in the immediate treatment of gastroesophageal reflux symptoms, where the volume of usual medications needs to be relatively high to achieve an acceptable duration of therapeutic effect, contrary to the present invention.

The composition of the present invention can be administered in the usual forms adapted to the mode of administration chosen, for example via the oral or parenteral routes, but preferably via the oral or intravenous routes. For example, it is possible to use tablet or capsule formulations containing tenatoprazole and the histamine h2-receptor antagonist as the active substances, or emulsions or solutions for parenteral use containing a tenatoprazole salt combined with one or more histamine H2-receptor antagonists, together with a standard, pharmaceutically acceptable substrate.

The unit doses may contain between 10 and 60 mg of tenatoprazole and between 40 and 400 mg of a histamine H2-receptor antagonist, particularly ranitidine or cimetidine.

As an example an appropriate formulation for capsules is shown below:

| | | |
|---|---|---|
| Tenatoprazole | 20 | mg |
| Ranitidine | 200 | mg |
| excipients | qs 300 | mg |

The dosage is determined by the practitioner as a function of the patient's state and the severity of the disorder. It is generally between 10 and 120 mg, and preferably between 20 and 40 mg tenatoprazole per day, with 200 to 400 mg of ranitidine.

For example, treatment for the nocturnal symptoms of gastroesophageal reflux may consist in the administration of 1 to 2 tablets, each containing 20 mg tenatoprazole and 300 mg ranitidine, to be taken every evening for a period which can range from 4 to 10 weeks, in the case of initial or maintenance therapy.

In patients with severe disorders, it may be effective to administer the medicinal product via the intravenous route in the first instance, and subsequently via the oral route. This invention also has the advantage of permitting sequential treatment which is effective using a single dose each week of one tablet containing 20 or 40 mg tenatoprazole combined with 20 to 300 mg of a histamine H2-receptor antagonist, such as ranitidine or cimetidine.

The study of clinical cases described below demonstrated the efficacy of the composition of this invention.

TABLE 2
Treatment of patients with symptoms of gastroesophageal reflux

| | Predominant | Duration of | Evolution of | |
|---|---|---|---|---|
| Age/Gender | symptom | treatment | symptom | Safety |
| 47/M | n.h. | 8 weeks | ++ | +++ |
| 47/F | n.h. | 8 weeks | +++ | +++ |
| 39/F | n.h. | 4 weeks | ++ | +++ |
| 32/F | n.h. | 8 weeks | +++ | ++ |
| 45/M | n.h. | 8 weeks | +++ | +++ |
| 50/F | n.h. | 8 weeks | +++ | ++ |
| 34/M | n.h. | 4 weeks | +++ | +++ |
| 38/F | n.h. | 8 weeks | ++ | +++ |
| 46/M | n.h. | 8 weeks | +++ | +++ |

n.h: nocturnal heartburn
The symbols +, ++ and +++ identify the evolution of the symptom and safety as being moderate, favourable and very favourable, respectively.

Treatment consisted in the daily administration, at bed-time, of one tablet containing 20 mg tenatoprazole and 300 mg ranitidine. Table 2 above shows that the treatment was perfectly tolerated in 7 out of 9 cases, and well tolerated in the other two patients, and that the evolution observed in symptoms was generally very favourable.

What is claimed is:

1. A pharmaceutical composition for the treatment of a disease related to gastric hyperacidity, said composition comprising tenatoprazole and one or more histamine H2-receptor antagonists.

2. The composition according to claim 1, wherein the histamine H2-receptor antagonist is selected from the group consisting of cimetidine, ranitidine, famotidine and nizatidine.

3. The composition according to claim 1, wherein the weight ratio between tenatoprazole and the histamine H2-receptor antagonist is between 1:30 and 1:2.

4. The composition according to claim 1, wherein said composition comprises between 10 and 60 mg of tenatoprazole and between 40 and 400 mg of a histamine H2-receptor antagonist.

5. The composition according to claim 1, wherein tenatoprazole is in the form of a potassium, magnesium, sodium or calcium salt.

6. The composition according to claim 1, wherein said composition is in a form for oral or parenteral administration.

7. The composition according to claim 2, wherein the weight ratio between tenatoprazole and the histamine H2-receptor antagonist is between 2:30 and 1:2.

8. The composition according to claim 2, wherein said composition comprises between 10 and 60 mg of tenatoprazole and between 40 and 400 mg of a histamine H2-receptor antagonist.

9. The composition according to claim 3, wherein said composition comprises between 10 and 60 mg of tenatoprazole and between 40 and 400 mg of a histamine H2-receptor antagonist.

10. The composition according to claim 2, wherein said composition is in a form for oral or parenteral administration.

11. The composition according to claim 3, wherein said composition is in a form for oral or parenteral administration.

12. The composition according to claim 4, wherein said composition is in a form for oral or parenteral administration.

13. The composition according to claim 5, wherein said composition is in a form for oral or parenteral administration.

14. A medicinal product useful for the treatment of a disease related to gastric hyperacidity, said product comprising tenatoprazole and at least one histamine H2-receptor antagonist.

15. A method of treating a disease related to gastric hyperacidity, said method comprising administering a medical product comprising tenatoprazole and at least one histamine H2-receptor antagonist.

16. The method of claim 15, wherein the disease related to gastric hyperacidity is gastric and duodenal ulcers and symptoms and lesions related to gastroesophageal reflux.

17. The composition according to claim 1, wherein the histamine H2-receptor antagonist is ranitidine.

18. The medicinal product of claim 14, wherein the histamine H2-receptor antagonist is ranitidine.

19. The method of claim 15, wherein the histamine H2-receptor antagonist is ranitidine.
